(19) United States
(12) Patent Application Publication
(10) Pub. No.: US 2017/0335336 A1
(43) Pub. Date: Nov. 23, 2017

(54) USE OF A SELECTABLE MARKER GENE IN SUGAR BEET PROTOPLASTS TRANSFORMATION METHOD AND SYSTEM

(71) Applicant: SESVanderHave N.V., Tienen (BE)

(72) Inventors: Guy WEYENS, Beersel (BE); Marc LEFÉBVRE, Jodoigne Souveraine (BE); Shivegowda THAMMANNAGOWDA, Zaventem (BE); Murielle LOMMEL, Itterbeek (BE)

(21) Appl. No.: 15/318,283

(22) PCT Filed: Jun. 12, 2015
(86) PCT No.: PCT/EP2015/063144

§ 371 Date: Dec. 12, 2016

(30) Foreign Application Priority Data
Jun. 12, 2014 (EP) PCT/EP2014/062288

(51) Int. Cl. C12N 15/82; A01H 1/04; C12N 9/88; A01H 1/02; C12N 15/52

(52) U.S. Cl. CPC C12N 15/821; C12N 15/8286; C12N 15/8285; C12N 15/8282; C12N 15/8281; A01H 1/02; C12N 15/8241; C12N 15/52; C12N 9/88; A01H 1/04; C12N 15/8279

(57) ABSTRACT

A method for the transformation of sugar beet protoplasts includes obtaining protoplasts from stomatal guard cells isolated from a sugar beet plant. The protoplasts are transformed with a nucleic acid construct including a nucleotide sequence of interest and a selection marker sequence. One or more ALS inhibitors at a concentration that is lethal to the in vitro culture of the protoplasts are applied to an in vitro culture of the protoplasts. Sugar beet plants are regenerated from the surviving protoplasts having integrated the nucleic acid construct including the sequence of interest and the selection marker sequence. The selection marker sequence is the mutated BvALS113 sequence carrying in its sequence a mutation at amino acid 113 position from Alanine to Tyrosine.

### FIELD OF THE INVENTION

The present invention is in the field of plants biotechnology, more particularly is related to a plant transformation method and means, especially to a method and means for transforming sugar beet protoplasts, cells, tissues (calli) and/or plants by using a new selection marker genetic sequence, preferably the mutated BvALS selectable marker genetic sequence.

The present invention is also related to a method for regenerating transgenic sugar beet plants from said transformed sugar beet protoplasts, cells or tissues and to transgenic plants and seeds obtained by such method.

### BACKGROUND OF THE INVENTION

Sugar beet (Beta vulgaris L.) is an important agricultural crop involved in about 20% of world sugar production. Despite increasing demand for genetically modified sugar beet plants, sugar beet plant is still a difficult plant to transform and a difficult material for plant regeneration. Improvement and innovation are still needed to obtain a reliable and efficient transformation and regeneration process of such plant.

As a general rule, and more particularly for plants which are difficult to transform, like sugar beet, the availability of appropriate effective means and method for selecting the transformed protoplast, cell or tissue is crucial.

Furthermore, as the selection marker is generally present in the transformed plant, the use of nucleotide sequences, like genes coding for resistance to an antibiotics are not accepted, because subsequent and often complicated and expensive methods should be used for removing the selection marker out of the transformed plant.

As sugar beet is an important agricultural crop in temperate and subtropical regions, herbicides are widely used to manage weeds proliferation during their growth. These new developed herbicides include Acetolactate synthase (ALS) also known as Acetohydroxyacid Synthase (AHAS) inhibitors. The genes coding for Acetolactate synthase (ALS) are known from U.S. Pat. Nos. 5013,659, 5,141,870 and 5,378,824.

### AIMS OF THE INVENTION

The present invention aims to provide a new method and new means for the transformation of sugar beet protoplasts, cells, tissues (calli) and plants that do not present the drawbacks of the state of the art, especially a method and means that are based upon the use of a new selection marker for improving the selection of genetically modified sugar beet protoplast, cells, tissues and plant comprising and expressing one or more gene of interest.

### SUMMARY OF THE INVENTION

The present invention is related to new method and new means for a transformation of sugar beet protoplast(s), cell(s), tissue(s) and plant(s) by using BvALS113 mutated genetic sequence as selection (selectable) marker gene.

Preferably, the present invention is related to a transformation method of sugar beet protoplasts, cells, tissues and/or plants with a nucleic acid construct (or vector comprising this nucleic acid construct) and to this nucleic acid construct comprising (or consisting of) a nucleotide sequence (preferably a gene) of interest and a selection marker sequence being the mutated BvALS113 sequence carrying in its sequence a mutation at amino acid 113 position from Alanine to Tyrosine, when compared to the wild type ALS sequence.

Preferably, in the nucleic acid construct, vector or method according to the invention, the nucleotide sequence of interest is selected from the group consisting of sequences encoding peptides conferring resistance to insects, nematodes or plant diseases or nucleotides, sequences encoding peptides against saline or hydric stress or sequences encoding one or more enzyme(s), antifungal peptides or antibacterial peptides, or a mixture thereof.

Advantageously, the nucleic acid construct according to the invention and used in the method of the invention is included into a vector that further comprises (adequate) regulatory sequences for expression of the nucleotide sequence of interest and possibly of the selection marker sequence in sugar beet protoplasts, cells, tissues or plants These regulatory sequences are preferably selected from the group consisting of promoters, transcription termination and/or poly-adenylation signal sequence(s) active in plants, more preferably the (CAMV) 35S promoter sequence and Nos terminator sequence (from Agrobacterium tumefaciens).

Preferably, the selection (selectable) marker sequence according to the invention is the sequence SEQ. ID NO: 3 comprising a mutation in its (wild-type) ALS sequence at amino acid 113 position (from an L-Alanine (Ala) to an L-tyrosine (Tyr)) when compared to its corresponding wild type sequence (being preferably the sequence SEQ. ID NO:1).

Advantageously, the method according to the invention comprises the following steps:
obtaining protoplasts from stomatal guard cells isolated from a sugar beet plant,
transforming these protoplasts with a nucleic acid construct (or vector) comprising the nucleotide sequence of interest and the selection marker sequence above described,
applying to an in vitro culture of these protoplasts, one or more ALS inhibitor(s) at a concentration that is lethal to (more than (about) 99.9%) these in vitro cultured protoplasts and regenerating one or more sugar beet plant(s) from the surviving protoplasts of these in vitro cultured protoplasts and wherein the surviving protoplasts being the one having integrated the nucleic acid construct comprising this nucleotide sequence of interest and this selection marker sequence (providing resistance to one or more ALS inhibitor(s)).

In the method according to the invention, the terms “more than (about) 99,9% of” mean more than 99%; 99.1%; 99.2%; 99.3%; 99.4%; 99.5%; 99.6%; 99.7% or 99.8%.

More advantageously, in the method according to the invention, the ALS inhibitor is applied at a concentration comprised between (about) 5×10⁻⁹M and (about) 1×10⁻⁸M for foramsulfuron, (about) 5×10⁻¹¹M and (about) 5×10⁻¹⁰M for ethoxysulfuron.

Suitable ALS inhibitors are preferably selected from the group consisting of sulfonylurea herbicides, sulfonylaminocarbonyltriazolinone herbicides, imidazolinone herbicides, triazolopyrimidine herbicides and pyrimidinyl(thio)benzoate herbicides. More preferably, the method according to the invention comprises the step of applying several ALS inhibitors present in a composition that comprises at least one sulfonylurea herbicide and at least one triazolopyrimidine herbicide.

The preferred ALS inhibitors are sulfonylurea herbicides selected from the group consisting of foramsulfuron (ALF), iodosulfuron, amidosulfuron, ethoxysulfuron (ALE), chloramsulfuron or a mixture thereof.

Other suitable ALS inhibitors are thiencarbazone-methyl and triazolopyrimidine herbicides.

The person skilled in the art may also select others adequate herbicides that interact with the above mentioned ALS sequence and wherein the above-mentioned mutation in SEQ. ID. NO: 3 (wherein there is an amino acid 113 position mutation from a L-Alanine (Ala) to a L-tyrosine (Tyr) compared to the wild type sequence SEQ. ID. NO: 1) may render protoplasts, cells and plant resistant to this ALS inhibitor or mixture of ALS inhibitors.

In the method according to the invention, a callus is the result of the growth of (well-regenerating) stomatal guard cells protoplasts.

Advantageously, the calli obtained by these (well-regenerating) protoplasts have the capacity to develop shoots and to regenerate into a viable sugar beet plant, when grown in a suitable culture media, such as polymer-containing medium (i.e. such as alginate or agarose containing medium).

In the method according to the invention, the ALS inhibitor is (or comprises) foramsulfuron, such as foramsulfuron applied to a one-week old (or to a three-weeks old) in vitro culture of protoplasts (more particularly to the in vitro culture comprising calli regenerated from these cultured protoplasts) on alginate-containing medium.

In the method according to the invention, the protoplasts are preferably transformed through agrobacterium-mediated process (or method) and the method preferably comprises also a step wherein the selection marker sequence is eliminated (removed from the plant genome) by crossing the transformed plants with a non-transformed variety of the same plant.

The present invention is also related a nucleic acid construct comprising (or consisting of) a nucleotide sequence (preferably a gene) of interest and a selection marker sequence being the mutated BvALS113 sequence carrying in its sequence a mutation at amino acid 113 position from Alanine to Tyrosine and to the vector comprising this nucleic acid construct and one or more regulatory sequence(s) for the expression of the nucleotide sequence of interest and possibly the selection marker sequence of this nucleic acid construct, into a sugar beet protoplast, cell, tissue and/or plant.

A last aspect of the invention is related to the protoplast(s), cell(s), tissue(s) (calli) or plant(s) obtained by the transformation method or comprising the nucleic acid construct or vector according to the invention, more particularly to a sugar beet plant integrating in its genome the nucleotide sequence of interest. The present invention will be described in details in the following examples in reference to the enclosed figures presented as non-limited embodiment of the present invention.

### SHORT DESCRIPTION OF THE DRAWINGS

FIG. 1 represents the pS189 vector according to the invention.

FIG. 2 represent sequences of BvALS gene, including the used sequence SEQ ID NO: 3.

FIG. 3 represents survivability of sugar beet protoplasts transformed by the pS189 vector of the invention.

### DETAILED DESCRIPTION OF THE INVENTION

Several sugar beet plant genotypes were selected for their capacity of regeneration from stomatal guard cells protoplasts. Protoplasts are selected by their capacity to growth and divide in vitro. A selection is made also upon the capacity of the grown calli to form shoots and the proportion of growing calli to regenerate a plant. Preferably, the selected genotype has more than 0.25% of the stomatal guard cells protoplasts that are able to grow in vitro. The person skilled in the art may, for instance, to refer to plants submitted to deposit as NCIMB 42050 or NCIMB 42051 as suitable genotype comprising a high proportion of growing stomatal guard cells protoplasts.

In the method according to the invention, the stomatal guard cells protoplasts, have the ability to divide (grow) and to regenerate (preferably via a sugar beet callus) when grown in a suitable culture medium. A callus refers to a mass of undifferentiated cells. In the art, a callus can be obtained from explants, such as embryos or parenchyma-derived explants from leaves or a cotyledon.

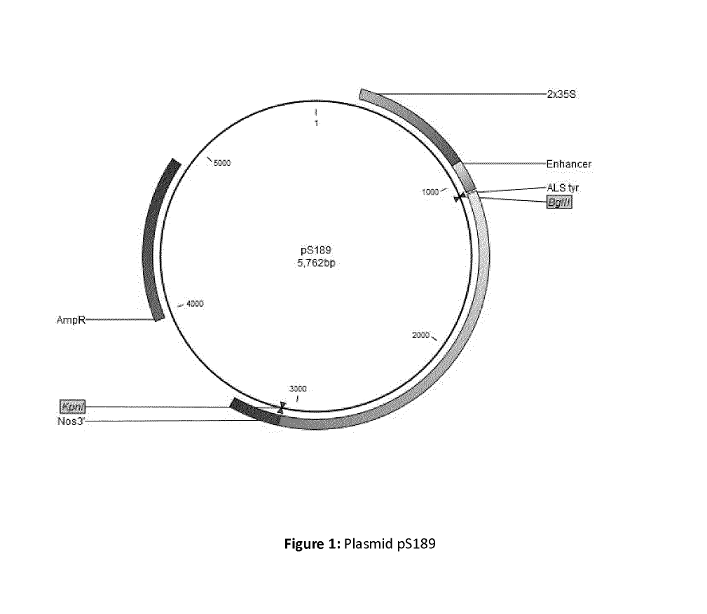

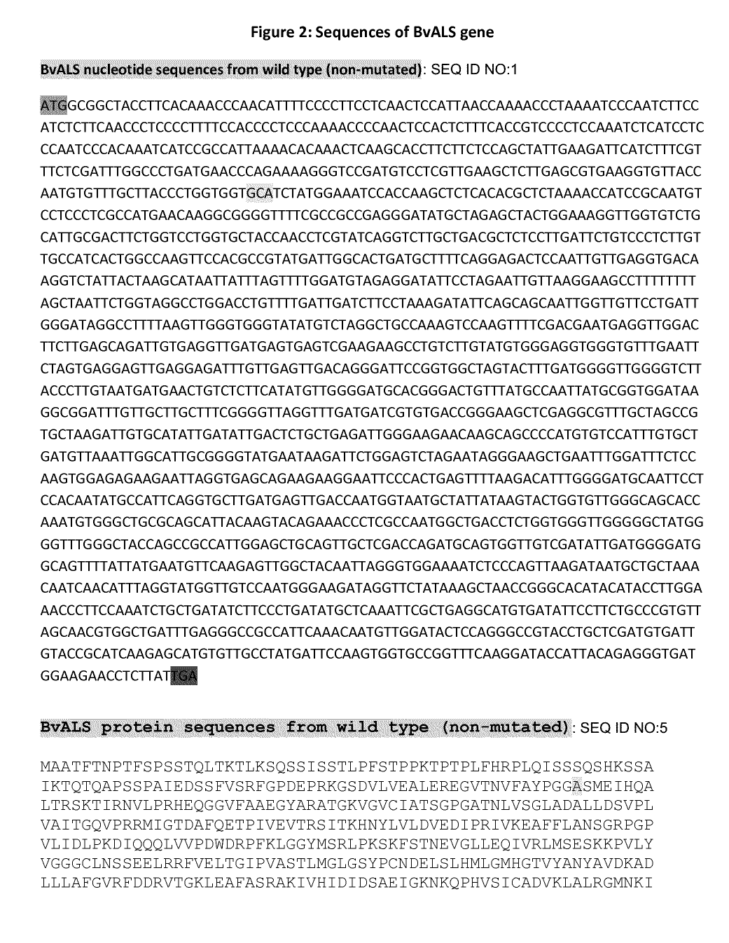

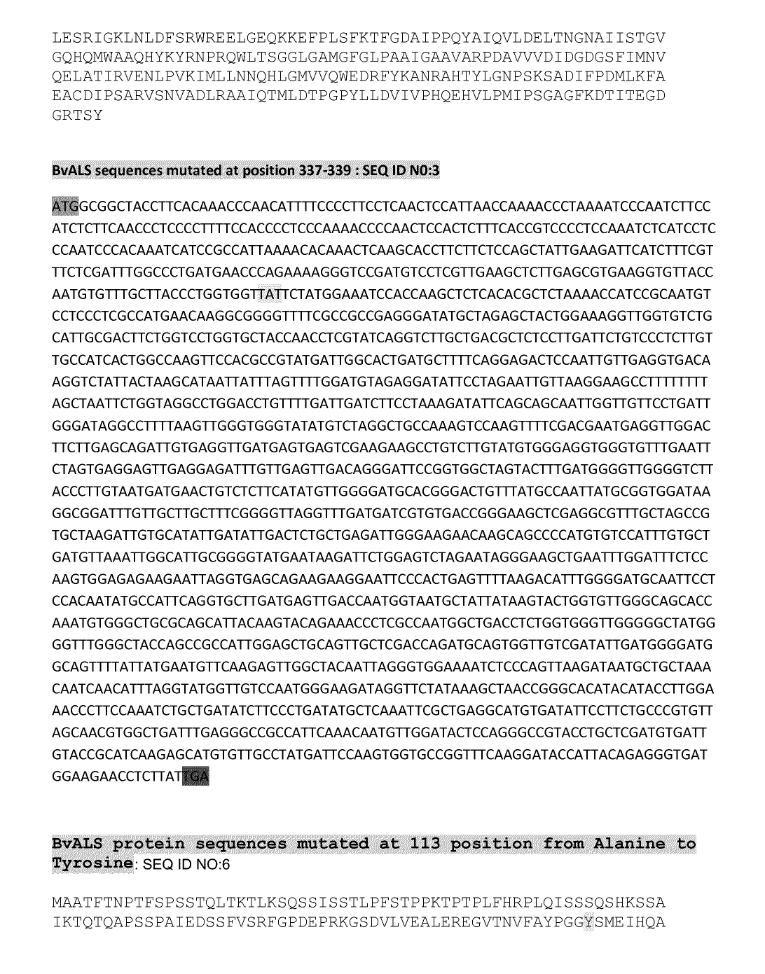

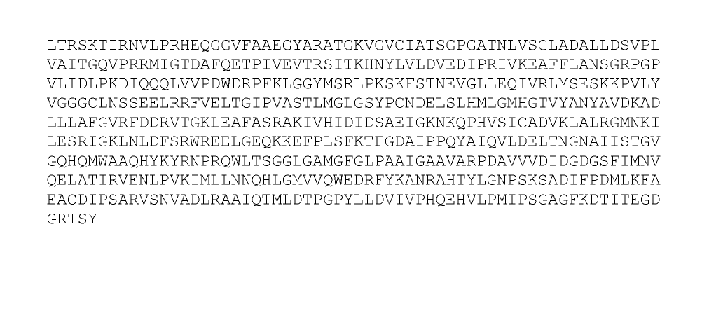

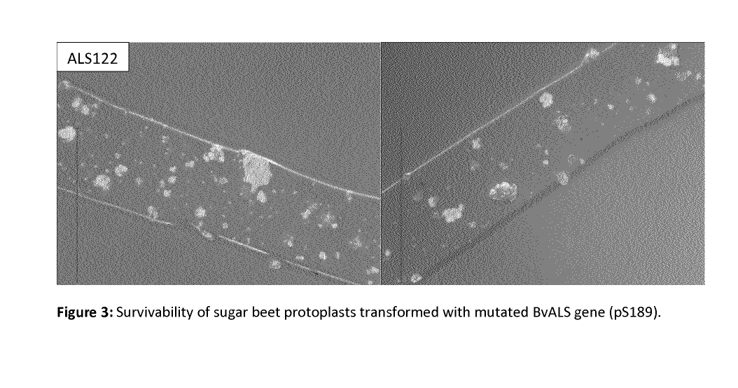

The used vector according to the invention is described in the enclosed FIG. 1 and may comprise a nucleic acid construct made of (comprising or consisting of) a nucleotide sequence of interest and the selection marker sequence of the invention both under the control of the constitutive (CAMV) 35S promoter and Nos terminator sequences. In this nucleic acid construct or vector according to the invention, the sugar beet ALS nucleotide sequence containing the nucleotides TAT at position 337 instead of GCA changing the corresponding amino-acid at position 113 from Alanine amino acid (Ala) to Tyrosine amino acid (Tyr) was synthetized by IDT (Integrated DNA Technologies) and inserted in the vector pIDT blue. The nucleic acid construct was then inserted as a KpnI-BgIII fragment into the pMJB3 plasmid between the 2×35S promoter for constitutive expression and the Nos terminator from Agrobacterium tumefaciens. The resulting plasmid (vector according to the invention) is named pS189 and represented in FIG. 1.

The optimal herbicide (ALS inhibitor) concentration used in the method according to the invention was determined from a killing curve concentration for two preferred ALS inhibitors (Foramsulfuron and Ethoxysulfuron) and was established as follows:
To use optimal concentration of herbicide for selection of transformed sugar beet protoplasts, killing curve was developed on wild type sugar beet protoplasts with the following concentrations (Table 1 and Table 2). Based on the results obtained from the killing curve experiments concentration of 5×10⁻⁹M for foramsulfuron and 5×10⁻¹¹M for ethoxysulfuron were selected for using with transformed sugar beet protoplasts with pS189 plasmid DNA carrying the mutated BvALS nucleotide sequence.

Transformation experiments were performed using plasmid DNA (pS189) according to the standard PEG transformation system under two selection media containing 5×10⁻⁹M foramsulfuron (ALF) and 5×10⁻¹¹M ethoxysulfuron (ALE) concentrations. The selection of only the transformed protoplasts were evidenced by the number of calli recovered from each transformation experiment. As a control experiment, non-transformed protoplasts were placed on foramsulfuron and ethoxysulfuron selection media that did not result in any calli or survival of cells. So far seven transformation experiments were performed for ALF and six experiments for ALE. From ALF experiments, about 1799 buds and 89 regenerated plantlets were obtained (Table 3).

These eighty nine primary transformation events have been screened for ploidy level and resulting in twenty seven diploid events. Further molecular analysis for the confirmation of presence of transgene by PCR, copy number analysis by taqman analysis and Southern blot analysis will be performed on these events.

Various transformation methods can be used for instance, PEG addition or Agrobacterium mediated transformation vector (Agrobacterium vector) to insert a heterologous nucleotide sequence into a protoplast or a cell susceptible to infection by Agrobacterium.

The nucleic acid construct or vector according to the invention preferably comprises a promoter, an encoding sequence comprising the nucleotide sequence of interest, preferably a gene product of interest as well as the selection marker sequence, as well as regulatory sequences such as poly-adenylation signal and transcription activation sequences (enhancer, such as the translation activator sequence of the Tobacco mosaic virus (TMV) or the Tobacco etch virus (TEV)) and other transcription terminator (enhancer) sequence. The person skilled in the art can select others suitable sequences for obtaining expression of the selection marker sequence and the nucleotide sequence of interest into the selected cell, tissue and plant.

(Constitutive) Promoter sequences may be obtained from plant or virus and comprise but are not limited to, the 35S or 19S promoter(s) of cauliflower mosaic virus (CAMV) or from the circovirus and promoters isolated from plant genes, or specific to seeds, such as Napin promoter, the phasaeolin promoter, the glutenin promoter, the helianthinin promoter, the albumin promoter, the oesosin promoter, the SAT1 promoter, the SAT3 promoter and inducible promoters, such the Pal promoter, the HMG promoter, RuBisCO promoter and promoter obtained from T-DNA gene of agrobacterium tumefaciens, such as the nopaline promoter and the mannopine synthase promoter.

Therefore, the present invention is also related to a vector suitable for transforming sugar beet plant cells (possibly using Agrobacterium-mediated process) and comprising at least the (heterologous) nucleic acid construct comprising or consisting of the sequence of interest and the selection marker sequence according to the invention.

Various Agrobacterium strains can be employed including, but not limited to, Agrobacterium tumefaciens and Agrobacterium rhizogenes. Suitable Agrobacterium tumefaciens strains including A208EHA101 and LBA4404 strains. Suitable strain of Agrobacterium rhizogenes including K599 strain.

The selection marker sequence can be introduced into the protoplast or cell simultaneously with the nucleotide sequence of interest, preferably upon the same vector and under the control of the same regulatory sequences (same Promoter), but could also be associated in convergent/divergent or collinear manner or through administration of two vectors used simultaneously for transforming plant protoplast or cell.

In the nucleic acid construct or vector according to the invention, the nucleotide sequence of interest is a gene encoding a protein of interest under the control of a regulatory sequences active into a plant cell (such as promoter sequence which is functional in the selected plant cell or plant of interest) to confer on the transformed plant novel agronomic properties or improvements in the agronomic quality of the transformed plant. Preferably, these sequences of interest are selected from the group consisting of sequences encoding protein(s) conferring resistance to certain insects, conferring resistance to nematodes, conferring resistance to certain diseases, sequences encoding specific enzymes and/or sequences encoding antibacterial or antifungal peptides or proteins.

1.-12. (canceled)

13. A method for transformation of sugar beets comprising the steps of:
obtaining protoplasts from stomatal guard cells from a sugar beet plant,
transforming said protoplasts with a nucleic acid construct comprising SEQ ID NO:3,
applying to an in vitro culture of said protoplasts, one or more ALS inhibitors at a concentration that is lethal to the protoplasts that do not express SEQ ID NO:4; and
regenerating sugar beet plants from surviving protoplasts.

14. The method of claim 13, wherein the nucleic acid construct comprises both a nucleotide sequence of interest and a selection marker sequence, SEQ ID NO:3 being either said nucleotide sequence of interest or said selection marker sequence.

15. The method of claim 15, wherein the nucleic acid construct further comprises one or more regulatory sequences for expression of the nucleotide sequence of interest and the selection marker sequence in sugar beet protoplast, cell, tissue and/or plant.

16. The method of claim 13, wherein the ALS inhibitor is applied to more than twenty million protoplasts.

17. The method of claim 13, wherein the ALS inhibitor is selected from the group consisting of sulfonylurea herbicides, sulfonylaminocarbonyltrazolinone herbicides, imidazolinone herbicides, triazolopyrimidine herbicides, pyrimidinyl(thio)benzoate herbicides, thiencarbazonemethyl herbicides or a mixture thereof.

18. The method of claim 17, wherein the sulfonylurea herbicide is selected from the group consisting of Foramsulfuron, iodosulfuron, amidosulfuron, ethoxysulforon, chloramsulfuron, or a mixture thereof.

19. The method of claim 18 wherein the ALS inhibitor is applied at a concentration between 5×10-9M and 1×10-8M for foramsulfuron, 5×10-11M and 5×10-10M for ethoxysulfuron.

20. The method of claim 13, wherein the protoplasts are transformed through an agrobacterium-mediated process.

21. The method of claim 13, wherein two nucleic acid constructs are applied for transformation of the protoplasts, wherein one nucleic acid construct comprises a nucleotide sequence of interest and the second nucleic acid construct comprises a selection marker sequence, said method further comprising eliminating the selection marker sequence by crossing transformed plants with a non-transformed variety of the same plant.

22. The method of claim 13, wherein the nucleotide sequence of interest encodes a peptide selected from the group consisting of peptide conferring resistance to insects, peptide conferring resistance to nematodes, peptide conferring resistance to plant diseases, peptides encoding an enzyme activity, antifungal peptides and antibacterial peptides.

23. A nucleic acid construct comprising a nucleotide sequence of interest and a sequence encoding SEQ ID NO:4.

24. The nucleic acid construct of claim 23, the nucleic acid construct being in the form of a vector for expression of the nucleotide sequence of interest into a sugar beet protoplast, cell, tissue and/or plant.

25. The nucleic acid construct of claim 24, wherein the vector further comprises one or more regulatory sequence(s), said vector being for expression of the nucleotide sequence of interest and of the selection marker sequence into a sugar beet protoplast, cell, tissue and/or plant.

26. A transgenic cell expressing SEQ ID NO:4.


SEQUENCE LISTING (4 sequences)

SEQ ID NO: 1 - DNA, 1998 bp, Beta vulgaris
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaatgggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattga

SEQ ID NO: 2 - protein, 665 aa, Beta Vulgaris
MAATFTNPTFSPSSTQLTKTLKSQSSISSTLPFSTPPKTPTPLFHRPLQISSSQSHKSSAIKTQTQAPSSPAIEDSSFVSRFGPDEPRKGSDVLVEALEREGVTNVFAYPGGASMEIHQALTRSKTIRNVLPRHEQGGVFAAEGYARATGKVGVCIATSGPGATNLVSGLADALLDSVPLVAITGQVPRRMIGTDAFQETPIVEVTRSITKHNYLVLDVEDIPRIVKEAFFLANSGRPGPVLIDLPKDIQQQLVVPDWDRPFKLGGYMSRLPKSKFSTNEVGLLEQIVRLMSESKKPVLYVGGGCLNSSEELRRFVELTGIPVASTLMGLGSYPCNDELSLHMLGMHGTVYANYAVDKADLLLAFGVRFDDRVTGKLEAFASRAKIVHIDIDSAEIGKNKQPHVSICADVKLALRGMNKILESRIGKLNLDFSRWREELGEQKKEFPLSFKTFGDAIPPQYAIQVLDELTNGNAIISTGVGQHQMWAAQHYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSKSADIFPDMLKFAEACDIPSARVSNVADLRAAIQTMLDTPGPYLLDVIVPHQEHVLPMIPSGAGFKDTITEGDGRTSY

SEQ ID NO: 3 - DNA, 1998 bp, Beta Vulgaris
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggttattctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaatgggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattga

SEQ ID NO: 4 - protein, 665 aa, Beta Vulgaris
MAATFTNPTFSPSSTQLTKTLKSQSSISSTLPFSTPPKTPTPLFHRPLQISSSQSHKSSAIKTQTQAPSSPAIEDSSFVSRFGPDEPRKGSDVLVEALEREGVTNVFAYPGGYSMEIHQALTRSKTIRNVLPRHEQGGVFAAEGYARATGKVGVCIATSGPGATNLVSGLADALLDSVPLVAITGQVPRRMIGTDAFQETPIVEVTRSITKHNYLVLDVEDIPRIVKEAFFLANSGRPGPVLIDLPKDIQQQLVVPDWDRPFKLGGYMSRLPKSKFSTNEVGLLEQIVRLMSESKKPVLYVGGGCLNSSEELRRFVELTGIPVASTLMGLGSYPCNDELSLHMLGMHGTVYANYAVDKADLLLAFGVRFDDRVTGKLEAFASRAKIVHIDIDSAEIGKNKQPHVSICADVKLALRGMNKILESRIGKLNLDFSRWREELGEQKKEFPLSFKTFGDAIPPQYAIQVLDELTNGNAIISTGVGQHQMWAAQHYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSKSADIFPDMLKFAEACDIPSARVSNVADLRAAIQTMLDTPGPYLLDVIVPHQEHVLPMIPSGAGFKDTITEGDGRTSY